(19) United States
(12) Patent Application Publication
(10) Pub. No.: US 2008/0275402 A1
(43) Pub. Date: Nov. 6, 2008

(54) FISTULA INSERT

(75) Inventor: Ralf Schnell, Seligenstadt (DE)

Correspondence Address: PAUL AND PAUL, 2000 MARKET STREET, SUITE 2900, PHILADELPHIA, PA, 19103, US

(73) Assignee: TRACOE MEDICAL GMBH, Frankfurt/Main (DE)

(21) Appl. No.: 12/109,456

(22) Filed: Apr. 25, 2008

(30) Foreign Application Priority Data
Apr. 27, 2007 (DE) 10 2007 021 111.4

(51) Int. Cl. A61M 16/04

(52) U.S. Cl. 604/175

(57) ABSTRACT

The present invention concerns a fistula insert with a tubular body which has a distal and a proximal end and a central lumen, in particular to seal fistulas in connection with voice prostheses, speech valves, tracheal cannulas, stoma buttons and connectors. To produce such a device, which on insertion can be conformed individually to the size and shape of the stoma or fistula and produce a suitable seal, in accordance with the invention, the tubular body has an outer surface which can be conformed to the shape of a fistula to be sealed and an inner surface which is essentially stable in shape.

The present invention relates to a fistula insert with a tubular body having a distal and a proximal end and a central lumen, in particular to seal fistulas in connection with voice prostheses, speech valves, tracheal cannulas, stoma buttons and connectors.

In tubular openings produced artificially or through disease or deformity, which constitute body openings or which run inside the body, termed fistulas or stomata, devices such as valves are often inserted to carry out a particular function. As a rule, this function is deleteriously affected or impeded if the device does not seal the fistula opening. Particularly with valves, in the event of leaks between the fistula and volume the possibility arises that liquids, gases or even solids can pass by the valve and thus deleteriously affect its function.

An example of a closure for a stoma is a stoma button. These buttons are intended to fit and seal the tracheostoma of a patient to as great an extent as possible. Furthermore, stoma buttons are usually equipped to connect auxiliary devices such as speech valves or artificial noses (heat moisture exchangers, HME). Such auxiliary devices can only function effectively if the stoma button seals the stoma sufficiently. If the seal is not sufficient, leaks would prevent voice production or effective moistening of the breath from the lungs.

Known stoma buttons exist in a wide variety of lengths and diameters. However, it is not possible to safely seal every stoma with known commercially available stoma buttons as the stomata of different patients vary widely in size and shape. Moreover, a fistula or a stoma is not stable and changes over time. Patients with oversized or very deformed stomata often fix HMEs or speech valves using self adhesive dressings with appropriate adapters. However, changing such dressings daily often gives rise to skin irritations at the stoma and is also very time consuming and awkward.

Even with so-called voice prostheses, a similar problem arises. These prostheses are inserted in patients who have had a laryngectomy where the larynx has been surgically removed and thus it is no longer possible to produce a natural voice. In these patients, a fistula is formed between the windpipe and the oesophagus, allowing air from the lungs to pass into the oral cavity. To prevent liquids or chyme from entering the lungs via the windpipe in this manner, a (one-way) valve, the so called voice prosthesis, is inserted into that fistula.

A similar problem arises with such voice prostheses, namely that leaks may occur between the fistula opening and the valve. This is not only very unpleasant for the patient, but also very dangerous as liquids or chyme can bypass the voice prosthesis, enter the windpipe and finally enter the lungs. A possible way to overcome this problem is to close the fistula up with a suture or to inject collagen into the tissue forming the fistula. Particularly with very deformed fistulas, however, such solutions are not always successful. Under some circumstances, then, in order to stop leaks the fistula must be closed and a new fistula must be formed at another location.

Even the use of voice prostheses with a very large diameter, which are literally wedged into the fistula and thus to a certain extent enlarge them, is no solution to the problem as too great a pressure of the voice prosthesis on the fistula kills the tissue surrounding the fistula and thus enlarges the fistula even further.

Many different voice prostheses are known which improve the seal by means of flanges at the ends or lamellae which lie within the fistula. However, even those voice prostheses cannot be conformed to the fistula immediately on insertion by the physician or the patient, so that a leak cannot be immediately corrected. In particular, irregularly shaped fistulas cannot be sufficiently seated using such voice prostheses as they are usually circular in cross section.

Thus, the aim of the present invention is to provide a device for sealing fistulas with voice prostheses, speech valves, tracheal cannulas or connectors such as stoma buttons which on insertion can be individually conformed to the size and shape of the stoma and provides a reliable seal.

This aim is achieved by dint of a fistula insert with a tubular body which has a distal end and a proximal end and a central lumen, wherein the tubular body has an outer surface which can be conformed to the shape of a fistula to be sealed, and an inner surface which is essentially stable in shape.

The term “inner surface” means the surface which delimits the central lumen. The term “conformable outer surface” is essentially the surface which comes into contact with the fistula and in particular with the fistula opening. Clearly, after insertion of the insert into the fistula the longitudinal axis of the central lumen runs essentially parallel to the longitudinal extent of the fistula opening.

The term “stable in shape” means that the shape of the central lumen is indeed deformable if a sufficiently high pressure is exerted on the insert, but for an inner or outer over pressure of 100 mbar, no substantial deformation occurs.

The central lumen of the tubular body can serve to hold a voice prosthesis, a speech valve, a tracheal cannula or a connector. Clearly, the shape-stable inner surface may consist of the outer walls of the inserted voice prosthesis, speech valve or connector. However, the central lumen can also remain open so that the insert serves only to hold the fistula open.

The lumen of the fistula insert can have a circular cross-section, but it may also have an oval, elliptical, angular or irregularly shaped cross section. Thus, the lumen does not necessarily lie exactly centrally in the cross section through the tubular body. In particular, with very deformed fistulas, the lumen may also be displaced from the centre, but positioning the inner surface and thus the lumen as centrally as possible is preferred.

The “proximal end” indicates that end which, following insertion into a fistula, comes to lie on the side from which the insert was inserted. When inserting fistulas which project outwards (external fistulas), the proximal end is thus the end which projects from the body. The “distal end” defines the opposite end of the tubular body.

Preferably, the distance of the conformable outer surface from the proximal end of the tubular body is at most 10 mm, preferably at most 8 mm and particularly preferably at most 5 mm. The fistula insert serves to hold fistulas open or to attach or insert any auxiliary devices into an external or internal fistula; it is constructed so that the conformable outer surface lies in the fistula opening and covers it. In this respect it is advantageous for the conformable outer surface to be as close as possible to the proximal end so that this latter projects only slightly from the fistula, thus reducing the risk of an irritation or injury to the fistula due to accidental movements.

In the case of fistula inserts for internal fistulas, both the proximal and the distal ends are advantageously a short distance from the conformable outer surface of at most 10 mm, preferably at most 8 mm and particularly preferably at most 5 mm.

Particularly preferably, for the reasons given above, the distance from the distal and proximal ends of the conformable outer surface of the tubular body should be as similar as possible so that it lies as centrally as possible on the tubular body. This encompasses embodiments wherein the distances vary by about 10%.

Preferably again, the tubular body is from 0.4 cm to 6 cm, preferably 0.6 cm to 4 cm long. For most artificially inserted fistulas, this length is sufficient to pass completely through the fistula opening and for the fistula insert to project from the fistula opening by as short a distance as possible.

In a particularly preferred embodiment of the fistula insert, the length of the conformable outer surface is greater than the length of the fistula opening of the fistula for which the insert is to be used. This means that the conformable outer surface forms a flange at one or both ends beyond the fistula to be sealed when dilating and/or when inserting the insert into the fistula. Such a flange covers the rim of the fistula completely on the appropriate side and lies close to the tissue surrounding the fistula. It serves to fix the fistula insert in the fistula. In addition, it makes it difficult for liquids or solids, in particular chyme components or secretions, to get between the rims of the fistula between the fistula and the insert.

Further, it is also possible that independently of the conformable outer surface, flanges which are essentially stable in shape are provided at the distal and proximal ends of the fistula insert, connected to the stable inner surface. These shape-stable flanges can, for example, ensure that on dilating, the conformable outer surface does not become extended in the direction of the central opening and close it. In this respect, the radial extent of the shape-stable flanges should be small enough so that on feeding them through the fistula, stretching of the fistula does not occur or substantially does not occur. At one end, the proximal end, which is not to be fed through the fistula, the flange can clearly have a larger extent.

If the fistula ends in a tubular opening, the diameter of which essentially corresponds to that of the fistula and which constitutes an extension to the fistula, as is the case, for example, with the windpipe which following a laryngectomy ends in an opening in the skin, in accordance with one embodiment the insert can have a smaller flange at the end which reaches into the passage. This smaller flange can also consist only of the shape-stable fistula described above which is independent of the conformable outer surface.

Advantageously, the flange or flanges of the fistula insert are preformed in the conformable outer surface in its undilated condition and/or before inserting the insert into the fistula. In this respect, the preformed flanges are advantageously also conformable so that after inserting the insert into the fistula or after dilating the insert, they fit closely to the rims of the fistula and seal them.

Further, the flanges, together with or separately from the tubular body, may also be dilatable and thus can be conformed to the rims of the fistula.

In a preferred embodiment, the outer surface consists of a closed-pore foam. It is also possible for both the inner and the outer surfaces or for the whole surface of the tubular body to consist of a closed-pore foam.

The term “surfaces formed from closed-pore foam” means a closed cell foam which covers only the surface of the fistula insert.

However, the fistula insert can also consist of an integral foam wherein a closed cell outer skin encloses an open-cell core. Mixtures of different types of foam may also be envisaged. However, the outer surface must in all cases consist of a deformable soft foam so that this surface can be conformed to the shape of the fistula.

In particular, the foam ensures that the insert conforms well to the shape of the fistula to be sealed. The closed-pore outer surface thus prevents the ingress of liquid or solids, in particular chyme, into the insert and simultaneously fits the fistula so closely that neither liquids nor solids can penetrate between the fistula and the insert. Even the passage of a gas through the insert or between the insert and fistula can be prevented by such a foam. In this respect, the foam should be soft enough to exert a pressure of less than 30 mbars on the tissue.

In a preferred embodiment, at least the outer surface of the fistula insert consists of an outer skin which together with the inner surface delimiting the central lumen defines a volume which can be dilated and wherein the outer surface, by dilating the volume, can be matched in shape to the fistula to be sealed.

The advantage of this embodiment lies in the fact that on dilating, the volume can be adjusted so that the conformable outer surface comes into contact with and seals the whole circumference of the fistula opening and thus seals the fistula.

Further, a material can be used for the outer surface which essentially serves to prevent the ingress of liquids, solids or gases into the insert and thus is capable of fitting the fistula closely. The material with which the insert is dilated can essentially have properties which allow a particularly even pressure to be exerted by the insert on the fistula and which actually accomplishes the task of creating a seal between the fistula and the insert.

In the case of dilatable fistula inserts, the pressure on the fistula wall can be adjusted by adjusting the dilating quantity. The pressure which is exerted by the insert on the fistula wall should be sufficient to satisfy the demands placed on the insert. A balance must be struck between a pressure which is sufficiently high to ensure that the fistula insert is sealed, and in particular to prevent a patient from aspirating the insert, and a sufficiently low pressure to prevent the tissue around the fistula from being damaged. The most important factor in cases of doubt is to keep the tissue intact.

The outer skin can extend only over the conformable outer surface or over further parts or the whole surface of the insert as long as the outer skin and other components of the insert or the speech valve, voice prosthesis or connector create a volume which can be dilated.

Particularly preferably, the outer skin consists of a thin foil which can delimit the volume without its thickness contributing too greatly to the volume. Clearly, this foil must be tolerated by skin and tissue. The foil essentially acts as a carrier for transferring the properties, in particular pressure, of the dilating material to the wall of the fistula. Furthermore, it can itself seal the insert from its surroundings. The foil thickness should thus not be less than 5 μm, not exceed 2 mm and preferably be between 75 and 900 μm.

Particularly preferably, the foil is formed from a flexible and/or elastic material. These materials are particularly capable of matching themselves to the shape of a fistula. The possibility of the foil stretching with an elastic material can be of particular assistance when sealing very large fistulas. In this case the insert should be dilated so that only a moderate pressure is exerted on the fistula, in particular the fistula opening, the fistula rims and the surrounding tissue, so that the tissue does not necrotize. The foil must thus match the contour of the fistula and stretch even at low pressures of less than 100 mbar, preferably less than 50 mbar, particularly preferably between 15 and 30 mbar. The resulting low resistance of the foil to stretching allows the pressure exerted on the tissue surrounding the fistula to be determined more precisely, since the stretching resistance of the foil counters the pressure exerted by the dilating.

When using an elastically extensible foil, various embodiments of the fistula insert may be envisaged. The fistula insert could, for example, already be dilated prior to insertion. In this case, its external diameter prior to insertion is larger than the diameter of the fistula opening. On insertions the fistula insert is compressed across its cross-section and the elastically extensible foil contracts in these regions and thus lies against the fistula opening without creasing.

In order for the elastically extensible foil to fit the fistula opening without creasing, it is necessary for the fistula insert to be dilated until the outer skin has no creases but is not yet stretched and has an external diameter which is smaller than the diameter of the fistula or corresponds thereto.

For certain embodiments, foils which are not extensible or are plastically extensible in certain pressure ranges may be envisaged as the material for the foil.

The foils may thus be formed from various materials or mixtures of materials. Examples of suitable materials are polyurethane, polypropylene, polyethylene, polyethylene terephthalate, polyvinyl chloride or other polymers and polymer blends as well as SEBS (styrene-ethene-butene-styrene), SBS (styrene-butadiene-styrene), SIS (styrene-isoprene-styrene), IR (polyisoprene) or other thermoplastic elastomers, latexes, silicones, natural rubbers or synthetic rubbers.

Preferably again, the volume delimited by the outer skin can be dilated with a fluid or a gas. Both liquids and gases distribute the pressure of the insert on the fistula evenly through the outer skin so that a particularly good seal can be achieved. The insert could also be dilated with a gel with the same properties.

Particularly preferably, the volume may be dilated with a liquid which can harden. Examples of such liquids are resins or rubbers which polymerize by a chemical reaction inside the insert. Examples are both single- and multi-component systems, for example formed from polyurethane or silicone.

Materials which solidify at about 42° C. to 55° C. may also be envisaged so that in the liquid state they are at a temperature which is tolerable to the patient and does not result in tissue destruction. Examples in this regard are types of paraffin, stearin, beeswax or polyethylene glycol, for example PEG 1000 PEG 1500, PEG 2000 or PEG 4000 or mixtures thereof. At body temperature, these materials solidify or are in their solid aggregate state.

A crystallization process within the insert may also be envisaged. In this case, the volume is dilated with a supersaturated melt. By producing or releasing crystallization seeds, the material can be solidified in a controlled manner by initiating crystallization. An example of a possible material with such properties which could be used for the dilating is sodium acetate trihydrate.

An essential advantage of the use of materials which solidify or crystallize is that warming it up again re-initiates the deformability of the material so that it can be conformed again by heating and cooling.

The solidified materials are thus preferably elastically or plastically deformable to a small extent so that the insert can be removed, for example for cleaning, by compressing it without stretching the surrounding tissue too greatly. On the other hand, the flexibility should be low enough for the insert to seal the fistula tightly.

In a further preferred embodiment, the insert is dilated with a foam. Advantageously, it is a soft foam which can be deformed under low pressure. Ingress of liquids or chyme into the foam is prevented in this embodiment by the outer skin. As an example, the foam can be formed by a chemical reaction taking place after dilating the insert if it is already in the fistula. With such a soft material, however, it is also possible for the foam to be placed in the volume delimited by the outer skin on manufacture of the insert and on inserting the insert into the fistula, a seal is produced by the slight elastic deformability of the foam. The large number of cells or chambers which constitute such a foam which can be compressed independently of each other means that such a loam can readily be conformed to irregularly shaped fistulas.

With inserts dilated with soft foam which are delimited by a foil, it is also possible to remove air which is in the foam before insertion into the fistula, thus reducing the volume and rendering insertion easier. After positioning the fistula insert in the fistula, the air can be let back in, in which case the insert practically dilates itself and thus is pressed tightly against the fistula and in particular the fistula opening.

Particularly preferably, the voice prosthesis, speech valve or connector is connected with the insert by adhesion or welding. In this case, stable types of connection are used which simultaneously produce a seal between the insert and the corresponding device in the central lumen. However, the voice prostheses, speech valves or connectors may be screwed into the central lumen with the help of a screw thread. Advantageously again, the central lumen is formed as a tube with a connector onto which voice prostheses or speech valves can be attached.

In a further preferred embodiment of the invention, the outer surface of the insert is provided with a lubricant. In this manner, introduction of the insert into the fistula is facilitated. In order to prevent the insert from sliding out easily, a water-soluble lubricant may be used which when inside the body only remains on the outer surface of the insert for a short period. Such a lubricant may, however, also be applied to the surface for the sole purpose of introducing the insert.

Advantageously again, the pressure exerted by the seal on the fistula wall can be adjusted so that too high a pressure on the tissue surrounding the fistula and any possible necrosis resulting therefrom is avoided. Such an adjustability is possible by dint of selecting the type of foam and the diameter of the insert with respect to the diameter of the fistula for which the insert is to be used; the pressure on the fistula wall must be about 10 to 25 mbar.

Particularly preferably, the external diameter of the region of the tubular body which comes to lie inside the fistula and fits the fistula opening dilated is larger than the diameter of the fistula when it is not constricted externally. In particular, such an embodiment of the insert produces a good seal even with a very low pressure of the insert on the fistula opening and thus on the surrounding tissue.

The outer surface of the insert can thus lie in very small creases on the fistula opening which, however, because a suitable material has been selected for the surface, have such small dimensions that penetration of liquids or gases through the creases is prevented. In particular, when using thin foils as the material for the outer skin, the foil thickness should be selected so that it is between 5 μm and 150 μm, preferably between 10 and 70 μm, whereby sufficient mechanical stability is ensured for sufficient flexibility. The foil is thus essentially not extensible so that the pressure which is exerted on the tissue surrounding the fistula can be determined directly by measuring the internal pressure on dilating.

The stability of this embodiment can be improved by dilating with a hardenable liquid.

Further advantages characteristics and applications of the present invention will become clear from the description below of preferred embodiments made with reference to the accompanying drawings.

They show:

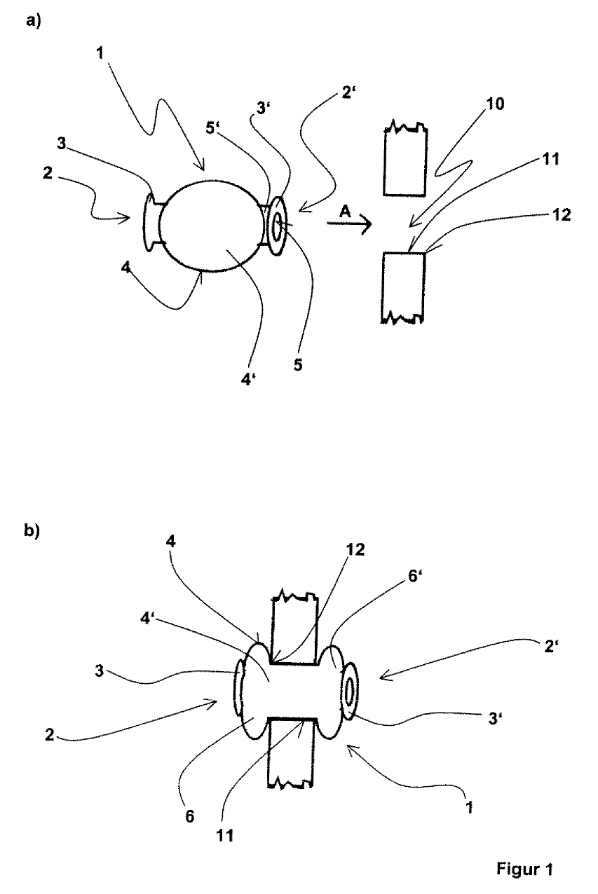

FIG. 1a: a diagrammatic representation of a longitudinal section through a fistula and a fistula insert of the invention prior to insertion;

FIG. 1b: a diagrammatic representation of a longitudinal section through a fistula and a fistula insert of the invention of FIG. 1a following insertion;

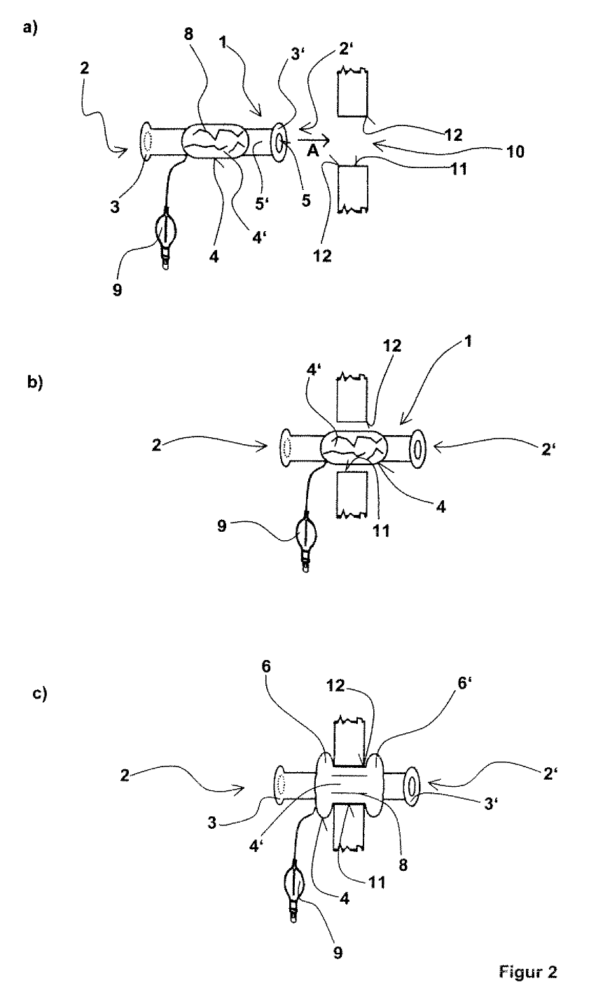

FIG. 2a; a diagrammatic representation of a longitudinal section through a fistula and another embodiment of a fistula of the invention prior to insertion;

FIG. 2b: a diagrammatic representation of a longitudinal section through a fistula and the fistula insert of FIG. 2a of the invention following positioning in the fistula; and

FIG. 2c: a diagrammatic representation of a longitudinal section through a fistula and the fistula insert of the invention of FIG. 2a following dilating.

FIG. 1a shows a diagrammatic view of a fistula insert 1 of the invention with a proximal end/and a distal end 2′. It has an outer surface 4, which can be conformed to the shape of the fistula 10 to be sealed.

In the embodiment of the invention shown in FIG. 1, the outer surface 4, which can be conformed to the shape of the fistula 10 to be sealed, is formed by a body 4′ formed from foam. It is essentially an open pore foam which is delimited by a gas- and liquid-impermeable outer skin which in fact forms the conformable surface. The outer skin may, for example, be formed from a closed-pore foam. However, a thin, flexible elastic foil may also be used, which is formed from PU, polypropylene, PE, polyethylene terephthalate, PVC, SEBS, SBS, SBS, IR, natural or artificial rubber, latex, silicone or mixtures thereof or materials with similar properties which are tolerated by body tissues.

The body 4′ forming the conformable outer surface extends around an essentially shape-stable inner surface 5 which is formed by a tubular body 5′. The tubular body 5′, which constitutes the inner surface, projects at both ends 2 and 2′ beyond the body 4′ forming the outer surface 4. At both ends 2 and 2′, flanges 3 and 3′ have been preformed which have an external diameter which, however, is not larger than the internal diameter of the fistula 10 which is defined by the fistula rims 12 and the fistula opening 11.

Arrow A indicates the direction along which the fistula insert 1 is inserted into the fistula 10.

FIG. 1b shows the fistula of FIG. 1a inserted in the fistula 10. For simplicity, the fistula is shown in longitudinal section. However, the insert is shown in side view, as shown in FIG. 1a.

The deformability of the body 4a with the outer surface 4 means that the fistula insert 1 can be introduced into the fistula 10 without widening the fistula rim 12 or the fistula opening 11. For this reason, compression of the material of the body 4′ which is under the outer surface 4 forms flanges 6 and 6′ either side of the fistula. The preformed flanges 3 and 3′ on the body 5′, which are essentially stable in shape like the inner surface 5, prevent the material of the body 4′ under the outer surface 4 from being displaced too far in the direction of one of the ends 2 or 2′. This avoids complete or partial closing of the central lumen of the tubular body of the fistula insert 1 by the conformable surface 4.

Flanges 6 and 6′ lie either side of the fistula 10 close to the fistula rims 12. Simultaneously, the surface 4 has conformed to the fistula opening 11, wherein the material of the beds 4′ is partially con pressed and thus fits the fistula opening 11 and exerts a pressure thereon. By selecting a suitable material and a suitable diameter for the body 4′ so that it can crease freely, the pressure of the outer surface 4 on the fistula opening 11 is about 10 to 25 mbar, so that a sufficient seal is produced and at the same time the pressure which is exerted on the tissue is not so high that it could result in tissue damage. With such fistula inserts 1, additional control of the pressure is superfluous.

In other possible embodiments which correspond to the fistula inserts shown in FIGS. 1a and 1b, the body 4′ is dilated with a material which can harden. This produces a body which is essentially stable in shape the surface of which, following hardening, is no longer directly conformable. Examples of filler materials are those which can harden reversibly, such as a polyethylene glycol (PEG) which has a hardening temperature of more than 40° C., or a supersaturated melt, or those which can harden irreversibly by polymerization, wherein the material is selected so that at least a slight elastic or plastic deformability is still possible. Examples of such polymerizing materials are single- or multi-component silicon rubbers or polyurethane resins.

In the embodiment which is described above which is dilated with foam, the outer skin of the body 4′ which forms the outer surface 4 is formed by a thin, flexible elastically extensible foil. This foil is extended across its section on introducing the fistula insert and thus fits against the fistula rims and the fistula opening without creasing.

The fistula insert 1 with hardened bodies 4′ are introduced into the fistula 10 in a deformable state. Hardening is carried out when the insert is in the fistula as shown in FIG. 1b. In this respect, the filler material is selected so that hardening occurs after a certain delay so that any corrections to the seat of the insert 1 in the fistula 10 or the distribution of the material on flanges 6 and 6′ can still be made.

Fistula inserts with reversibly hardening bodies 4′ can easily be removed from the fistula 10 by re-liquefying or re-establishing the deformability of the materials. With irreversibly hardening fistula inserts, removal of the insert is made possible by dint of the slight elastic or plastic deformability.

FIG. 2a shows a fistula insert 1 which has a tubular body with an inner surface 5 and an outer surface 4 which can be conformed to the contour of a fistula. Here, the inner surface 5 is formed by a tubular body 5′ which has a proximal end 2 and a distal end 2′ and is partially surrounded by a tubular body 4′ which forms the conformable surface 4. The tubular body 45 is essentially centrally located on the length of the tubular body 5′, can be dilated and in the non-dilated condition shown in FIG. 2a is creased, as at 8. To fill the body 4′, a valve 9 is provided. When dilating the body 47 to the free uncreased condition, as shown in FIG. 1a, the outer diameter of the body 4′ would become large than the internal diameter of the fistula 10. The body 5′ is thickened in a flange-like manner at its ends 3 and 3′, which act as stabilizers and are provided for the attachment of speech valves and/or heat moisture exchangers.

The fistula insert 1 can be inserted into the fistula 10 in the direction of arrow A.

FIG. 2b shows the fistula insert 1 of FIG. 2a being inserted into the fistula. The outer diameter of the fistula insert 1 including the outer surface 4 is thus, both in the region of the fistula rim 12 and also in that of the fistula opening 11, smaller than the internal diameter of the fistula 10. The tubular body 4′ is still in the undilated condition and is creased irregularly at 8. The outer skin of the body 4′, which constitutes the conformable surface 4, consists of a highly flexible soft foil which also may be slightly extensible. It is acceptable to tissue and both gas- and liquid-impermeable.

The body 4′ can be dilated through the valve 9. Various gases, including air or a fluid can be introduced. The advantage of dilating in this manner is that to remove the fistula insert 1 from the fistula 10, it suffices to remove part of the gas or fluid via the valve 9. It is, however, also possible to introduce a hardening material. Suitable examples of hardening materials have been described above. Such fistula inserts dilated with a hardening, material are, in contrast to the inserts dilated with gas or a fluid wherein the valve 9 remains on the body 4′, also suitable for internal fistulas as the valve 9 which would harm the internal workings of the body can be removed from the body 4′ after the material has hardened.

FIG. 2c shows the dilated fistula insert 1 which corresponds to the insert of FIGS. 2a and 2b. Since the length of the body 4′ and thus the conformable surface 4 is greater than the length of the fistula opening 11, flanges 6 and 6′ have been formed at both ends of the body 4′. Since on dilating without external constrictions, the body 4 has a larger external diameter than the internal diameter of the fistula 10 for which the fistula insert is to be used, when the body 4, has been dilated, the conformable surface 4′ is creased at the fistula opening and the fistula rim. The greater flexibility of the foil means that these creases are very small, however, so the crease dimensions are very small and the fistula is tight to gas and liquid around the fistula insert.

The flanges 6 and 6′ which form on dilating fit the fistula rim closely and the tissue surrounding the fistula. The distance between these flanges formed by dilating and the thickened parts 3 and 3′ at the ends 2 and 2′ amounts in this embodiment to less than 1 cm, usually less than 0.5 cm. Thus, the danger of injury by the projecting ends of the body 5, formed by the inner surface 5 is minimized, which is particularly important for inserts for internal fistulas. Even with external fistulas, the distance to the proximal end of the insert in particular should not exceed the value given above so that the insert does not project unnecessarily far out of the body.

In the embodiments of FIGS. 1 and 2, the body 5′ which is formed by the inner surface can consist of a stoma button or a voice prosthesis. The body 4′ is thus connected by welding or with adhesive.

Other possible embodiments can be envisaged wherein a voice prosthesis is attached to the inner surface 5 by welding or adhesion inside the tubular body 5′.

For the purposes of disclosure, it is stated herein that any features which the skilled person discerns from the present description, drawings and claims, even when described explicitly only in connection with other particular features, unless expressly excluded or technically impossible or without purpose, can be combined individually and in any combination with other features or groups of features disclosed herein. A comprehensive explicit description of all possible combinations of features is not given here for the purposes of conciseness and legibility of the description.

1. A fistula insert (1) with a tubular body which has a distal (2′) and a proximal (2) end and a central lumen, wherein the tubular body has an outer surface (4) which can be conformed to the shape of a fistula (10) to be sealed, and which has an inner surface (5) which is essentially stable in shape, in particular to seal fistulas in connection with voice prostheses, speech valves, tracheal cannulas, stoma buttons and connectors.

2. A fistula insert (1) according to claim 1, characterized in that the distance between the proximal end (2) of the tubular body and the conformable outer surface (4) of the tubular body is at most 10 mm, preferably at most 8 mm, particularly preferably at most 5 mm.

3. A fistula insert (1) according to claim 1 or claim 2, characterized in that the conformable outer surface (4) of the tubular body is essentially centrally arranged between the two ends (2, 2′).

4. A fistula insert (1) according to one of the preceding claims 1-2, characterized in that the tubular body is 0.4 cm to 6 cm long, preferably 0.5 cm to 4 cm long.

5. A fistula insert (1) according to one of the preceding claims 1-2, characterized in that the length of the conformable outer surface (4) is longer than the length of the fistula opening (11) of the fistula (10) for which the insert (1) is to be used.

6. A fistula insert (1) according to one of the preceding claims 1-2, characterized in that prior to introducing the insert (1) into the fistula (10), a flange (6, 6′) is preformed at one or both ends in the conformable outer surface (4).

7. A fistula insert (1) according to one of the preceding claims 1-2, characterized in that at least the conformable outer surface (4) is delimited by a closed-pore foam.

8. A fistula insert (1) according to one of claims 1 to 2, characterized in that the conformable outer surface (4) is formed by an outer skin which delimits a volume which can be dilated and on dilating, its external shape can be conformed to a fistula (10) to be sealed.

9. A fistula insert (1) according to claim 8, characterized in that the outer skin consist of a thin foil.

10. A fistula insert (1) according to claim 8, characterized in that the foil is formed from a flexible and/or elastic material.

11. A fistula insert (1) according to one of the preceding claims 1-2, characterized in that the insert can be dilated with a fluid or a gas.

12. A fistula insert (1) according claim 11, characterized in that the fluid is a hardenable liquid.

13. A fistula insert (1) according to claim 11, characterized in that the fluid consists of a material selected from the group formed by paraffin, stearin, beeswax. PEG 1000, PEG 1500. PEG 2000 PEG 4000 and sodium acetate trihydrate.

14. A fistula insert (1) according to one of claims 1 to 2, characterized in that the insert is dilated with a foam.

15. A fistula insert (1) according to one of the preceding claims 1-2, characterized in that a connection with the voice prosthesis, speech valve, tracheal cannula and connector is produced by adhesion or welding.

16. A fistula insert (1) according to one of the preceding claims 1-2, characterized in that the outer surface is provided with a lubricant.

17. A fistula insert (1) according to one of the preceding claims 1-2, characterized in that the pressure exerted by the outer surface (4) on the fistula wall (11) is adjustable.

18. A fistula insert (1) according to one of the preceding claims 1-2, characterized in that the external diameter of the region of the tubular body which will lie inside the fistula and fits the fistula opening, when dilated with no external constraints, is larger than the diameter of the fistula for which the insert is to be used.
